(19) United States
(12) Patent Application Publication
(10) Pub. No.: US 2020/0368473 A1
(43) Pub. Date: Nov. 26, 2020

(54) Larissa's Tracheostomy Holder

(71) Applicant: Ricardo Davila, (US)

(72) Inventor: Shirley Tatiana Reyes Atoche, Palmdale, CA (US)

(21) Appl. No.: 16/135,794

(22) Filed: Dec. 7, 2018

(51) Int. Cl. A61M 16/04; A61M 25/02

(52) U.S. Cl. CPC A61M 16/0465; A61M 2025/026; A61M 2025/022; A61M 25/02

(57) ABSTRACT

A tracheostomy holder to keep a tracheostomy tube secure in place. The tracheostomy holder has two contoured straps for applying through a tracheostomy tube eyelet. The tracheostomy holder is designed to be used with children and adults with any type of neck (short neck, long neck) also helps to prevent sliding and skin irritation. The tracheostomy holder keeps skin dry reducing the risk of skin breakdown around the neck. The tracheostomy holder has two straps wherein at the end of each strap, is a rough Velcro, that attaches to soft Velcro in the center back of the tracheostomy pad. The center of the pad is cushioned to provide more comfort for the neck.

### BACKGROUND

Tracheostomy holder are straps that go around the neck to help hold the tracheostomy tube in place.

### SUMMARY

Larissa's Tracheostomy holder are made with high quality cotton fabric to avoid irritation and discomfort.

ENVIRONMENTALLY FRIENDLY because the tracheostomy holder is washable and reusable per same person.

Made with Latex free materials.

100% secure with high quality of loop and hook fastener straps.

Designed in different colors, helping people to feel emotional better. Our tracheostomy back pad is cushioned to provide comfort for the neck.

### DRAWING DESCRIPTION

FIGS. 1-4 are a front view of the tracheostomy holder.

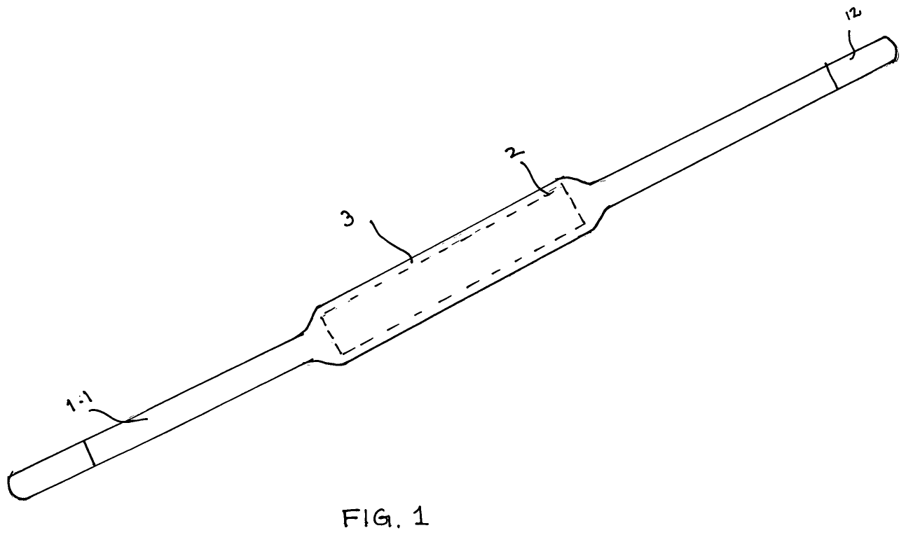

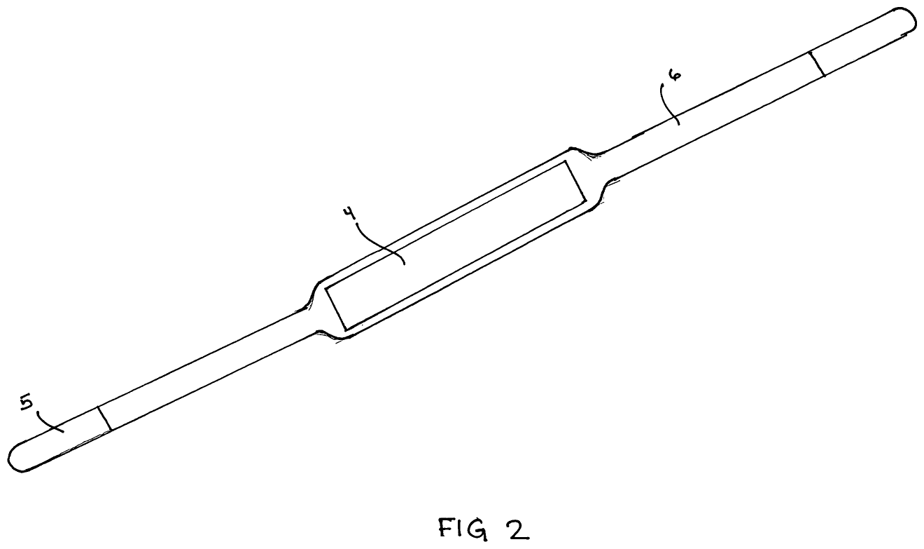

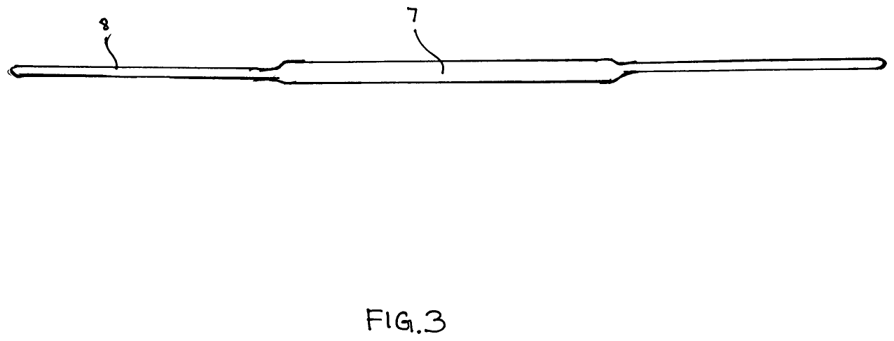

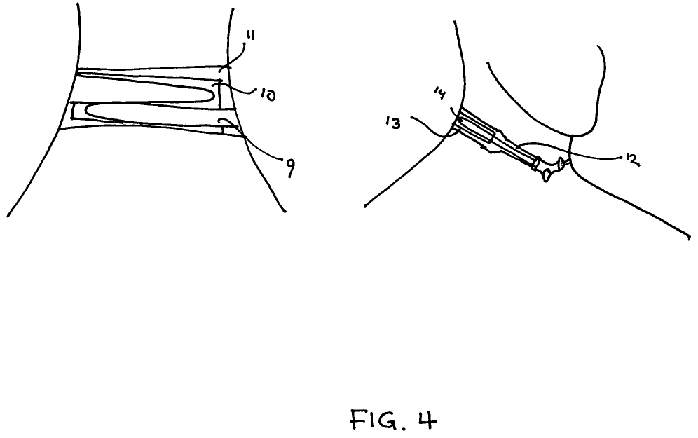

The following listing of numerals are present in this document:

1.1 shows the fabric strap.

2 is a stick of cotton thread.

3 shows the back cushioned pad of the tracheostomy holder.

4 is a loop ubicate in the back of the tracheostomy holder.

5 is the end of the strap which is the hook part.

6 is a back vid of the fabric strap.

7 shows the horizontal view of the tracheostomy pad.

8 is the horizontal part of the hook.

9 shows the hook attach on the loop part place on the tracheostomy holder on the back of the neck.

10 is the loop attach to the fabric pad on the back of the neck.

11 Tracheostomy holder back view.

12 shows the tracheotomy holder strap.

13 shows the tracheostomy pad from the side view.

14 shows the hook attach to the loop the go around the neck.

### DESCRIPTION

01—Larissa's Tracheostomy Holder is designed to provide COMFORT AND SAFETY to people who have Tracheostomy tube.

02—The Tracheostomy holder helps to keep tracheostomy tube secure in place. It is easy to put ON and REMOVE.

03—The tracheostomy holder comes with two contoured straps to mad it easy to apply through tracheostomy tube eyelets.

04—The tracheostomy holder is designed to be used with children and adults with any type of neck (short neck, long neck). Also helps to prevent sliding and skin irritation.

The tracheostomy holder keeps the skin DRY reducing the risk of skin breakdown around the neck.

05—Comes with two straps at the end of each straps is a rough hook fastener that will attach to the loop in the center back on the Tracheostomy pad and in which the center pads is cushioned to provide more comfort on the neck.

### Description:

Larissa's Tracheostomy holder is designed to provide COMFORT, and SAFETY to people who have Tracheostomy tube. The Tracheostomy holder is to keep tracheostomy tube secure in place. It is easy to put ON and Removed. The tracheostomy holder comes with two contoured straps to made it easy to apply, through tracheostomy tube eyelet. The tracheostomy holder is designed to be use with children and adults with any type of neck (short neck, long neck) also helps to prevent sliding and skin irritation. The tracheostomy holder will also keep skin dry reducing the risk of skin breakdown around the neck. The tracheostomy holder comes with two straps at the end of each straps is a rough velcro, that will attach to the soft velcro in the center back of the tracheostomy pad and in which the center pad is cushioned to provide more comfort for the neck.

Specification:

Larissa's Tracheostomy holder are made with high quality cotton fabric to avoid irritation and discomfort.
Environmentally friendly because the tracheostomy holder is washable and reusable per same person.
Made with latex free materials.
100% secure with high quality of velcro straps.
Designed in different colors, helping people to feel emotional better, made in all sizes.
The tracheostomy holder is made with cotton fabric, organic cotton, cotton thread, felt and velcro.

1- Larissa's Tracheostomy holder is a REUSABLE AND WASHABLE holder, made with high quality of materials.

2- tracheostomy holder made with quality of cotton thread and fabric flexible, soft and easy to put ON AND REMOVAL.

3- Tracheostomy holder back-pad extra cushioned for more comfort on the neck.
Larissa Tracheostomy holder as set forth in claim 1 due to the high quality of material this tracheostomy holder can be used up to 1 year guarantee.
